(19) United States
(12) Patent Application Publication
(10) Pub. No.: US 2014/0377195 A1
(43) Pub. Date: Dec. 25, 2014

(54) POLYMER COMPOSITION CONTAINING NATURAL BIOACTIVE PRINCIPLES FOR USE IN PHARMACEUTICAL AND COSMETIC FORMULATIONS

(71) Applicant: BIOATIVE TECNOLOGIA EM POLIMEROS LTDA-ME, São Paulo - SP (BR)

(72) Inventors: Walter Israel Rojas Cabrera, Sao Paulo - SP (BR); Karina Nakajima, Sao Paulo - SP (BR); Luana Bendo, Sao Paulo - SP (BR)

(21) Appl. No.: 14/368,243

(22) PCT Filed: Dec. 20, 2012
(86) PCT No.: PCT/BR2012/000520

§ 371 Date: Jun. 23, 2014

(30) Foreign Application Priority Data
Dec. 22, 2011 (BR) PI 1106762-4

(51) Int. Cl. A61K 36/18; A61K 8/86; A61Q 11/00; A61K 8/97; A61K 47/34; A61K 45/06

(52) U.S. Cl. CPC A61K 36/18; A61K 47/34; A61K 45/06; A61Q 11/00; A61K 8/97; A61K 8/86
USPC 424/58; 424/725; 424/769; 424/739; 424/756; 424/744; 424/745

(57) ABSTRACT

The present invention relates to a polymer composition containing natural bioactive principles, in particular propolis extract. The present composition is of value because it exhibits antimicrobial (antibacterial, antifungal, antiviral and antiprotozoal), wound healing, anti-inflammatory, antioxidising and moisturising properties. The composition according to the present invention can be used in pharmaceutical and cosmetic formulations, thus being useful for odontological treatments (of caries, gingivitis, periodontitis, bad breath, aphthae, oral stomatitis) and for the treatment of affections of the skin and mucous membranes. The present composition also contains biocompatible and biodegradable polymers that allow controlled release of the active principles. For this purpose, the product can be provided in the form of different formulations: spray, mouthwash, toothpaste, oral-base cream, lotions, creams, gels, inter alia.

### FIELD OF APPLICATION

The present invention relates to a polymer composition containing natural bioactives, in particular propolis extract. The present composition is important for presenting antimicrobial properties (antibacterial, antifungal, antiviral and antiprotozoal), as well as healing, anti-inflammatory, antioxidant and moisturizing properties.

The composition of the present invention can be used in pharmaceutical and cosmetic formulations, thereby being useful in dental treatment (caries, gingivitis, periodontitis, bad breath, canker sores, dental stomatitis), and cutaneous and mucosal diseases.

This composition also contains biocompatible and biodegradable polymers that allow controlled release of active principles. To this end, the product may present in different formulations: spray, mouthwash, toothpaste, Orabase ointment, lotions, creams, gels, among others.

### BACKGROUND OF THE INVENTION

### Description of the Prior Art

Currently, the use of natural products in pharmaceutical and/or cosmetic formulations has intensified due to the presence of active compounds with proven efficacy. Either in the extract form or by isolation of the active compounds, plant-derived products have been shown to be a viable alternative in the treatment of diseases when compared to allopathy. One of the widely studied compounds, propolis, has antimicrobial, anti-inflammatory, antioxidant, antitumor and healing properties, which makes it targeted to treat infections, among them infections related to the oral, skin and mucous membranes. Propolis consists of a resinous substance collected by honey bees of the genus Apis from different types of plants. Its composition may vary according to region and time of collection, but generally one can find approximately 50% of plant resins, 30% of wax (product of the metabolism of bees), 10% of essential and aromatic oils, 5% of pollen and 5% of other substances, including organic debris. According to Martos et al. (2008), propolis contains more than 300 active compounds, mainly phenolic compounds L0, including flavonoids (flavonols, flavones, flavonones and dihydroflavonones), derivatives of cinnamic acid and coumarin, as well as terpenes, sesquiterpenes and aromatic compounds. The main active compounds in propolis are described in Table 1. Due to the wide variety of biological actions, the use of propolis has been proposed in different dental treatments such as dental caries, gingivitis, periodontitis, bad breath, canker sores and dental stomatitis, and cutaneous and mucosal diseases.

Propolis has antibacterial activity due to the presence, among other compounds, of pinocembrine, galangin, CAPE, quercetin, apigenin and tt-farnesol, which act by increasing the permeability of the bacterial membrane or inhibiting enzymes essential for the function of the bacterium. Thus, formulations containing propolis extracts can be effective in the treatment of caries and periodontitis which are caused by bacterial infections. Streptococcus mutans consists of the main etiological agent of dental caries, being involved in the formation of bacterial plaque or biofilm. This consists of a multi-variety of bacteria which may cause demineralization of teeth (enamel, dentin and cementum) due to acid formation as a product of bacterial fermentation. Gingivitis (gum inflammation) also follows this process, and its evolution may result in periodontitis, a condition in which the gum and bone tissue are affected and may lead to tooth loss. In addition to the antimicrobial action, propolis has antioxidant and anti-inflammatory activity that may help in the treatment of oral diseases, including idiopathic stomatitis. Among the main anti-inflammatory agents, one can mention galangin, CAPE and chrysin, which work by suppressing certain inflammatory mediators, such as COX-1 and COX-2, iNOS and arachidonic acid.

Propolis is also effective against other microorganisms such as fungi, viruses and protozoa. In fact, the extract of propolis may help treat infections caused by Candida albicans and Herpes simplex, which affect not only the oral mucosa but also the genital region. Thus, the use of propolis extract may cover the treatment of genital and skin infections. In relation to skin tissue, however, studies have demonstrated the efficacy of propolis also in the treatment of burns, allergies and wound healing.

Due to its pharmacological properties, several pharmaceutical and/or cosmetic formulations containing propolis extract have been proposed as an alternative for the treatment and prevention of oral, skin and mucosa diseases. In dentistry, formulations containing propolis extract may also be used in the cleaning of cavities, in pulpectomy, surgical extraction procedures and dental implants, among others, in order to promote recovery.

TABLE 1
Active compounds present in the propolis extract

| Group | Class | Components |
|---|---|---|
| Flavonoids | Flavonols | Quercetin, kaempherol, galangin |
| | Flavones | Apigenin, acacetin, chrysin, |
| | | luteolin |
| | Flavonones | Pinocembrine, sakuranetin, |
| | | isosakuranetin |
| Compounds | Cinnamic | Caffeic acid phenethyl ester |
| Phenolic | acid | (CAPE), Artepelin C |
| Terpenes | | Carvacrol, tt-farnesol, geraniol, |
| | | cafestol, ledol, cimene, limonene, |
| | | estireno, naphthlene, β-bisabolol, |
| | | 1,8-cineol, clerodane derivatives, |
| | | labdane derivatives, β-amirin, |
| | | cembrene, squalene, β-caryophyllene, |
| | | pinene, taxadiene, lycopene. |

### Existing Problems

Chlorhexidine, triclosan, thymol and cetilpiridinium chloride are widely used in dental treatments as antimicrobial agents. In particular, chlorhexidine digluconate is the primary agent used in the treatment and prevention of oral disease due to its effectiveness against microorganisms, including bacteria (Gram-positive and Gram-negative), fungi, and viruses. However, the routine use of chlorhexidine is not recommended due to local adverse effects such as discoloration of teeth and tongues, pain and irritation of oral mucosa taste buds.

The prior-art documents PI0901470-5-1 and PI0502111 describe formulations containing dental and topical chlorhexidine in combination with propolis extract and other herbal compounds.

However, besides the presence of the drug, which has the above mentioned adverse reactions, the use of alcoholic extract of propolis may irritate the oral mucosa. This occurs because alcohol has the characteristic of promoting local dehydration, which could undermine the recovery of injured tissue.

Prior-art document PI0506243-8 proposes the use of hydro-alcoholic extracts of propolis in dental formulations which may present minor irritation; however, alcohol is still present. Likewise, the prior-art document PI0804023-0 makes use of alcoholic extracts to develop its formulations. In order to use propolis extract for treating burns and in dental, veterinary and cosmetic use, document PI0806114-9 refers to the development of products containing Poloxamer copolymer having the capacity to gel in contact with the skin, which promotes the adherence of the product to the tissue. However, this restricts the pharmaceutical presentation to a single form (sol-gel) and requires the storage of the product at low temperatures.

The prior-art document PI0803475-3 refers to the exclusive use of propolis in the development of pharmaceutical compositions having anti-neoplastic activity, as well as anti-inflammatory, antimicrobial and wound healing action.

The prior-art document US2007/0140990A1, however, uses propolis extracts at low ratios (0.0001 to 3%) for the development of specific products for oral care in combination with other active compounds in order to maximize the beneficial effects of the natural product.

In general, the products available use propolis as an adjuvant in the treatment of oral diseases, and the main active agents have potential side effects. Moreover, the use of alcoholic or hydro-alcoholic extracts of propolis may impair treatment since such a compound is extremely detrimental to the skin and mucous membranes.

### Objects of the Invention

In the present invention, the problems described above were resolved based on the modification in the composition of the formulations. Initially, the primary antimicrobial, anti-inflammatory, antioxidant and healing agent are natural products, especially non-alcoholic propolis extract, and the resulting formulations are applicable for the treatment and prevention of oral, skin and mucous diseases since the absence of alcohol favors their unrestricted use. Furthermore, the presence of biocompatible and biodegradable polymers contributes to local hydration because these are highly hydrophilic wetting agents, and are muco-adhesive which allows for greater adhesion to the application site. Another property of the polymer, which also renders the present invention innovative, is its ability to preserve constituents of propolis and provide better dispersion of the compounds due, in part, to the propolis encapsulation by the polymer. Thus, the present invention provides products with local and moisturizing action intensifying the action of the propolis active components which render them more effective for the recovery and maintenance of tissue integrity.

The detailed description that follows assists in the understanding of the product described in the present patent application; however, in non-limiting way, since optimizations in the process are acceptable.

### DESCRIPTION OF DRAWINGS

FIG. 1: Photomicrograph of spray formulation of 5% propolis demonstrating the variability in size of the propolis capsules formed by the polymer. 20× increase.

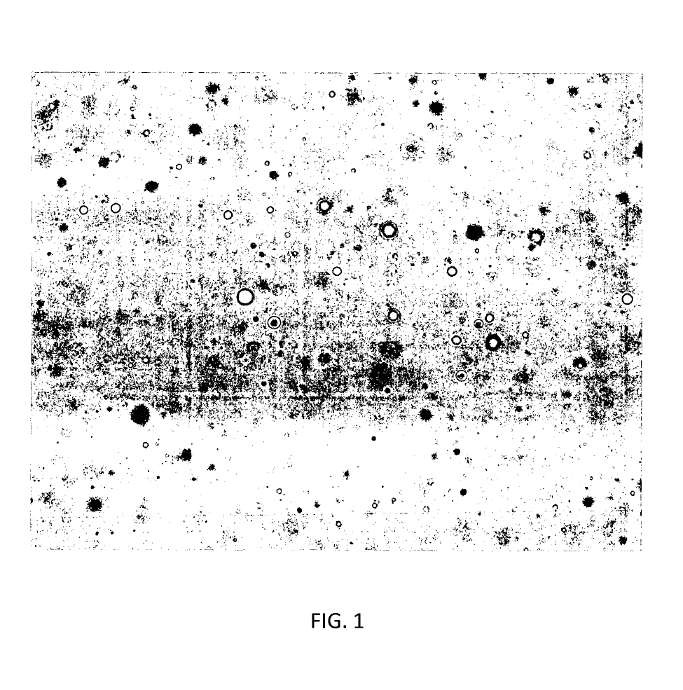

FIG. 2: Photomicrograph of spray formulation of 5% propolis demonstrating propolis encapsulation by the polymer. Capsule in detail (arrow). 40x increase.

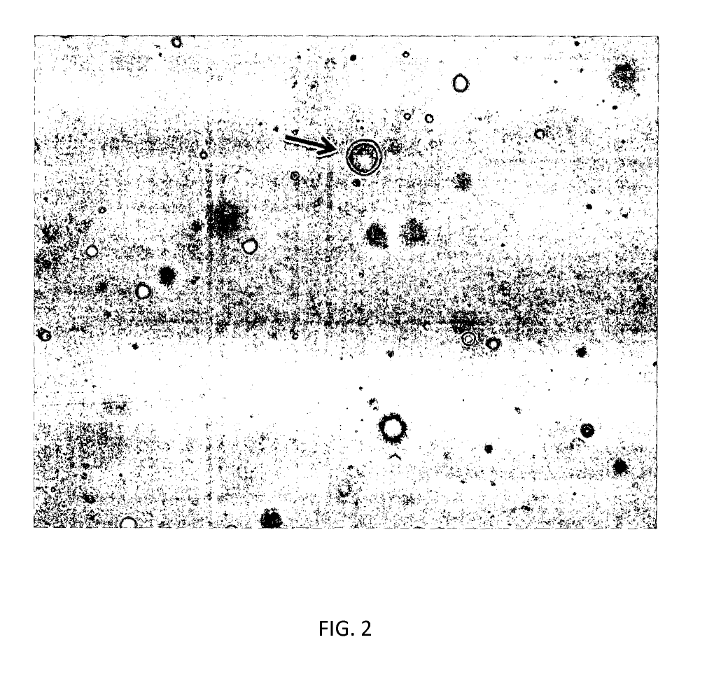

### DETAILED DESCRIPTION OF THE INVENTION

The present invention describes compositions based on propolis extract for use in pharmaceutical and cosmetic formulations, and may be useful in the treatment and prevention of oral, skin diseases and mucous diseases. The presentation form may be spray, mouthwash, toothpaste, Orabase ointment, lotions, creams, and gels, among others.

The present composition contains non-alcoholic propolis extract at a ratio of 1% to 30% (w/w), preferably 3% to 10%, being the main anti-inflammatory, antimicrobial, antioxidant and healing agent. Other natural compounds with anti-inflammatory and/or antimicrobial and/or wound healing and/or antioxidant properties may be introduced at appropriate concentrations, as formulation adjuvants, including Magnolia officinalis extract at 0.1% to 10% (w/w), Cinnamomum zeylanicum at the ratio of 5% to 30% (w/w), Calendula officinalis at the ratio of 0.3% to 10% (w/w), Zingiber officinale at a ratio of 2% to 5% (w/w), Aloe vera at the ratio of 2% to 20% (w/w), Melissa officinalis at the ratio of 0.5% to 10% (w/w), and others.

As biodegradable and biocompatible polymer, polyethylene glycol (PEG), Carbopol, Poloxamer may be used, among others. These are wetting and muco-adhesive agents, and favor the dispersion of the propolis constituents homogeneously. The polymer may be added at the ratio of 30% to 99% (w/w), preferably from 50% to 70%.

Other wetting agents may be included in the formulation as adjuvants, among them, sorbitol, glycerin and propylene glycol at a ratio of 1% to 60% (w/w), preferably from 10% to 30%.

The inclusion of flavoring agents renders the formulation more palatable, favoring its oral use. Natural or artificial flavoring agents may be used as aromatic oils (mint, spearmint, eucalyptus, among others) at a ratio which may range from 0.05% to 5% (w/w).

In addition to the flavoring agents, sweeteners, especially those that do not favor the formation of biofilms, may be added. Among some acceptable sweeteners for use in oral formulations there are saccharin, sucralose, stevia, xylitol, sorbitol, mannitol, aspartame, cyclamates and acesulfame. Such compounds may be added at the ratio of 0.1% to 5% (w/w), preferably from 0.5% to 2%.

Depending on the product presentation, thickening or gelling agents such as guar gum, xanthan gum, gum arabic, and gelatin, carboxymethylcellulose, sodium alginate and agar may be added, at the ratio which may vary from 0.3% to 5% (w/w).

Additionally, the oral compositions may include additives such as: brighteners, anticalculus and cariostatic agents. As brighteners, peroxides (hydrogen peroxide, calcium peroxide, urea peroxide, among others) may be used, and may be present in the formulation at a ratio of 0.01% to 10% (w/w), preferably from 0.1% to 5%. As a cariostatic agent, soluble fluoride ions are used (e.g., sodium fluoride) in an amount that may vary from 50 ppm to 4000 ppm, preferably from 500 ppm to 3000 ppm. As anticalculus agents, chelators are used, such as pyrophosphate and diphosphonate salts, which may vary from 1% to 15%.

It is also possible to use dyes (which can range from 0.001% to 0.5% (w/w)) to render the formulations more presentable.

As the formulation carrier water may be added at the ratio of 10% to 70% (w/w), especially from 30% to 60%.

Other ingredients may be added for improving formulations such as stabilizers, surfactants, emulsifiers, buffering agents and preservatives.

Here are some (not restrictive) examples of polymeric formulations containing natural bioactives:

### EXAMPLE 1

### Propolis spray 5%

| Component | Final Ratio |
|---|---|
| Non alcoholic propolis extract | 5% |
| Polyethylene glycol 400 (PEG) | 15% |
| Polyethylene glycol 1500 (PEG) | 28% |
| Saccharin | 1.5% |
| Menthol | 0.5% |
| Ultrapure water | 50% |

### EXAMPLE 2

### Propolis spray 3%

| Component | Final Ratio |
|---|---|
| Non alcoholic propolis extract | 3% |
| Polyethylene glycol 400 (PEG) | 15% |
| Polyethylene glycol 1500 (PEG) | 30% |
| Saccharin | 1.5% |
| Menthol | 0.5% |
| Ultrapure water | 50% |

### EXAMPLE 3

### Propolis Spray 1%

| Component | Final Ratio |
|---|---|
| Non alcoholic propolis extract | 1% |
| Polyethylene glycol 400 (PEG) | 15% |
| Polyethylene glycol 1500 (PEG) | 30% |
| Saccharin | 1.5% |
| Menthol | 0.5% |
| Ultrapure water | 52% |

### EXAMPLE 2

### Propolis ointment

| Component | Final Ratio |
|---|---|
| Non alcoholic propolis extract | 5% |
| Polyethylene glycol 400 (PEG) | 26.2% |
| Polyethylene glycol 1500 (PEG) | 55% |
| Saccharin | 1.6% |
| Menthol | 0.4% |
| Ultrapure water | 11.8% |

1. A polymeric composition comprising: non-alcoholic propolis extract at a ratio of 1% to 30% (w/w); biocompatible and biodegradable polymer at a ratio of 30% to 99% (w/w); and water at a ratio of 10% to 70% (w/w).

2. The polymeric composition of claim 1, wherein the polymer is selected from the group consisting of polyethylene glycol (PEG), Carbopol and Poloxamers.

3. The polymeric composition of claim 1, wherein the non-alcoholic propolis extract is at a ratio of 3% to 10% (w/w).

4. The polymeric composition of claim 1, wherein the polymer is at a ratio of 50% to 70% (w/w).

5. The polymeric composition of claim 1, further comprising natural compounds selected from the extracts of Magnolia officinalis at a ratio of 0.1% to 10% (w/w), Cinnamomum zeylanicum at a ratio of 5% to 30% (w/w), Calendula officinalis at a ratio of 0.3% to 10% (w/w), Zingiber officinale at a ratio of 2% to 5% (w/w), Aloe vera at a ratio of 2% to 20% (w/w), Melissa officinalis at a ratio of 0.5% to 10% (w/w), and combinations thereof.

6. The polymeric composition of claim 1, further comprising a wetting agent at a ratio of 1% to 60% (w/w).

7. The polymeric composition of claim 6, wherein the wetting agent is selected from the group consisting of sorbitol, glycerin and propylene glycol.

8. The polymeric composition of claim 6, wherein the wetting agent is at a ratio of 10% to 30% (w/w).

9. The polymeric composition of claim 1, further comprising a flavoring agent at a ratio of 0.05% to 5% (w/w).

10. The polymeric composition of claim 1, further comprising a sweetener at a ratio of 0.1% to 5% (w/w).

11. The polymeric composition of claim 10, wherein the sweetener is selected from the group consisting of saccharin, sucralose, stevia, xylitol, sorbitol, mannitol, aspartame, cyclamates and acesulfame.

12. The polymeric composition of claim 10, wherein the sweetener is at a ratio of 0.3% to 1% (w/w).

13. The polymeric composition of claim 1, further comprising a thickening or gelling agent at a ratio of 0.3% to 5% (w/w).

14. The polymeric composition of claim 13, wherein the thickening or gelling agent is selected from the group consisting of saccharin, guar gum, xanthan gum, gum arabic, gelatin, carboxymethylcellulose, sodium alginate and agar.

15. The polymeric composition of claim 1, further comprising a brightener at a ratio of 0.01% to 10% (w/w).

16. The polymeric composition of claim 15, wherein the brightener is selected from the group consisting of hydrogen peroxide, calcium peroxide and urea peroxide.

17. The polymeric composition of claim 15, wherein the brightener is at a ratio of 0.1% to 5% (w/w).

18. The polymeric composition of claim 1, further comprising a cariostatic agent in an amount that may vary from 50 ppm to 4000 ppm.

19. The polymeric composition of claim 18, wherein the cariostatic agent comprises soluble fluoride ions.

20. The polymeric composition of claim 18, wherein the cariostatic agent is in an amount that may vary from 50 ppm to 3000 ppm.

21. The polymeric composition of claim 1, further comprising an anticalculus agent at a ratio of 1% to 15% (w/w).

22. The polymeric composition of claim 21, wherein the anticalculus agent comprises a chelator.

23. The polymeric composition of claim 1, further comprising a dye at a ratio of 0.001% to 0.5% (w/w).

24. The polymeric composition of claim 1, further comprising a stabilizer, a surfactant, an emulsifier, a buffering agent, a preservative or a mixture thereof.

25. The polymeric composition of claim 1, wherein the composition is in the form of a spray, a mouthwash, a toothpaste, an Orabase ointment, a lotion, a cream or a gel.

26-27. (canceled)

28. The polymeric composition of claim 1, wherein the water is at a ratio of 30% to 60% (w/w).

29. The polymeric composition of claim 22, wherein the chelator comprises pyrophosphate or diphosphonate salts.

30. A pharmaceutical or cosmetic formulation for the treatment and prevention of oral, cutaneous and mucosal diseases comprising the polymeric composition of claim 1.
